(19) United States
(12) Patent Application Publication
(10) Pub. No.: US 2022/0373088 A1
(43) Pub. Date: Nov. 24, 2022

(54) DOOR SEAL

(71) Applicant: SciCan Ltd., Toronto (CA)

(72) Inventors: Stelian Gabriel GAL, Scarborough (CA); Bhupinder BHARAJ, Ajax (CA); Gabriel NEAGOE, Richmond Hill (CA); Christopher J. H. STEWART, Toronto (CA)

(21) Appl. No.: 17/327,402

(22) Filed: May 21, 2021

(51) Int. Cl. F16J 15/10; C09K 3/10; A61L 2/07; A61L 2/26

(52) U.S. Cl. CPC F16J 15/104; C09K 3/10; A61L 2/07; A61L 2/26; C09K 2200/0607; A61L 2202/121; A61L 2202/24

(57) ABSTRACT

Provided is a seal for sealing the interface between abutting first and second members, the seal comprising a main body; a channel lobe extending outward from the main body, the channel lobe shaped for nesting in a channel of the first member; a primary sealing lobe extending outward from the side of the main body facing the second member, the primary sealing lobe extending into and contacting an inner surface of the second member; and a secondary sealing lobe extending downward from the main body toward an exterior of the interface, a first face of a distal end of the secondary sealing lobe contacting a front face of the first member, and a second face of the distal end of the secondary sealing member contacting a front face of the second member. The disclosed seal maintains a gas-tight seal and minimizes resisting force when closing the door.

### FIELD OF THE INVENTION

The present invention relates to a seal to be used at the interface between two objections, such as the door of a pressure vessel and its pressurization chamber. The invention also relates to a pressure vessel that includes the seal.

### BACKGROUND OF THE INVENTION

Autoclaves are used to sterilize medical and dental instruments and equipment using steam. Typically, an autoclave will consist of a chamber, an access door, and a seal to achieve the elevated temperature and pressure that is required to destroy all pathogenic organisms to achieve sterility. As such, the chamber for the autoclave must be a gas-tight sealable pressure vessel able to withstand pressures seen during the sterilization cycle.

The door closing forces on manual door closures of tabletop autoclaves are often quite high. The sealing features of the autoclave door seal must typically be compressed enough to maintain a gas-tight pressure or vacuum seal in the autoclave chamber during operation. This door closing force can be overcome through mechanical methods or can be solved by modifying specific features in the door seal geometry.

International hygiene guidelines specify certain cycle profiles to obtain complete sterilization for the various instruments that require disinfection. These profiles often involve a conditioning phase that involves both air evacuation by way of vacuum and a heating and pressurization phase that involves the introduction of saturated steam. Autoclaves that have both vacuum and pressure profiles to adequately clean and sterilize equipment are designated as class B and class S type steam sterilizers.

Typical sterilization cycle profiles run at temperatures of 134 deg C. for 3-5.5 min. and 228 kPa. Class B and Class S type autoclaves use a repeated air removal and steam injection during the sterilization cycle profile. This positive and negative pressurization of the autoclave chamber creates additional constraints on the chamber seal to maintain a gas-tight enclosure.

A number of seals have been described in the art, for example, U.S. Pat. Nos. 10,436,326, 7,578,407, 3,334,774, 5,611,452, 7,413,099, 10,359,115, US 2009/0261533, U.S. Pat. No. 7,665,624, GB 2 282 422, and GB 919 432.

There is a need in the industry to produce a seal that maintains a gas-tight seal during pressurization and during vacuum, and that minimizes resisting force when closing the door.

### SUMMARY OF THE INVENTION

According to an aspect of the present invention, there is provided a seal for sealing the interface between abutting first and second members, the seal comprising: a main body; a channel lobe extending outward from the main body, the channel lobe shaped for nesting in a channel of the first member; a primary sealing lobe extending outward from the side of the main body facing the second member, the primary sealing lobe extending into and contacting an inner surface of the second member; and a secondary sealing lobe extending downward from the main body toward an exterior of the interface, a first face of a distal end of the secondary sealing lobe contacting a front face of the first member, and a second face of the distal end of the secondary sealing member contacting a front face of the second member.

In one embodiment, the main body is at least partially bifurcated, creating a slit between a portion of the channel lobe and a portion of the secondary sealing lobe.

In another embodiment, the first member and the second member are in a non-abutting state, the secondary sealing lobe is biased toward the primary sealing lobe.

In yet another embodiment, the seal further comprises an inner lobe extending outward from the main body toward the interior of the interface, a first face of the inner lobe contacting the front face of the first member. In a further embodiment, there is a depression in the main body at a transition point between the primary sealing lobe and the inner lobe.

In an embodiment, when the first member and second member are in an abutting state, the depression permits flexure of the primary sealing lobe toward the inner lobe.

In another embodiment, there are ribs and/or protrusions on a surface of one or more of the channel lobe, the primary sealing lobe, the secondary lobe, or the inner lobe.

In a further embodiment, the seal is made of an elastomeric material. In an embodiment, the seal is contiguous to seal a perimeter of the interface between the first and second members. The seal may have an annular shape, a substantially rectangular shape, or a substantially square shape. The primary sealing lobe may have a cone-shaped cross section.

According to another aspect, there is provided a seal for sealing the interface between abutting first and second members, the seal comprising: a main body; a channel lobe extending outward from the main body, the channel lobe shaped for nesting in a channel of the first member; a primary sealing lobe extending outward from the side of the main body facing the second member, the primary sealing lobe extending into and contacting an inner surface of the second member; a secondary sealing lobe extending downward from the main body toward an exterior of the interface, a first face of a distal end of the secondary sealing lobe contacting a front face of the first member, and a second face of the distal end of the secondary sealing member contacting a front face of the second member; and an inner lobe extending outward from the main body toward the interior of the interface, a first face of the inner lobe contacting the front face of the first member, wherein the main body is at least partially bifurcated, creating a slit between a portion of the channel lobe and a portion of the secondary sealing lobe; and there is a depression in the main body at a transition point between the primary sealing lobe and the inner lobe.

According to a further aspect, there is provided an autoclave comprising the seal as defined herein at an interface between a door and a pressurization chamber.

### BRIEF DESCRIPTION OF THE DRAWINGS

Reference will now be made, by way of example, to the accompanying drawings which show exemplary embodiments of the present application, and in which:

FIG. 1A is an illustration of an exemplary autoclave, in which the outer cover has been removed and the door is in the open position;

FIG. 1B is an illustration of an exemplary pressure vessel from the autoclave shown in FIG. 1A, in which the door is in the closed position;

FIG. 2A is a front view of a seal according to an embodiment of the invention;

FIG. 2B is a cutaway front perspective view of the seal shown in FIG. 2A;

FIG. 3A is a cross section view of a seal according to an embodiment of the invention;

FIG. 3B is a cross section view of a seal according to another embodiment of the invention, which is also an enlarged view of A in FIG. 2B;

FIG. 3C is a cross section view of a seal according to a further embodiment of the invention;

FIG. 4 is a cross section view of a pressure vessel with the seal shown in FIG. 3C sealing the interface between the closed door and the chamber;

FIG. 5 is an enlarged fragmentary view of the lower interface between the door and the chamber as shown in FIG. 4; and

FIG. 6 is a cross section view as shown in FIG. 5, in which the door is in the open position.

### DETAILED DESCRIPTION OF EXEMPLARY EMBODIMENTS

The features and benefits of the present disclosure are illustrated and described herein by reference to exemplary embodiments and is in no way intended to limit the invention, its application, or uses. This description of exemplary embodiments is intended to be read in connection with the accompanying drawings, which are to be considered part of the entire written description. Accordingly, the present disclosure expressly should not be limited to such embodiments, and features of each embodiment described herein may be combined with each other to form further embodiments.

In the description of embodiments disclosed herein, any reference to direction or orientation is merely intended for convenience of description and is not intended in any way to limit the scope of the present invention. Relative terms such as “horizontal”, “vertical”, “forward”, “backward”, “downward”, “outward”, “top”, “bottom”, “end”, derivatives thereof (e.g., “horizontally,” “downwardly,” “outwardly,” etc.), or any other terms related to direction or orientation should be construed to refer to the orientation as then described or as shown in the drawing(s) under discussion. These relative terms are for convenience of description only and do not require that the invention be constructed or operated in a particular orientation.

The invention generally relates to a multi-lobe seal. While the seal of the present invention is described with reference to a pressure vessel, such as an autoclave, it is to be understood that the seal may be used in any instance in which a seal between two structures is desired. For example, the seal of the present invention could also be used at the interface between a cap and a container.

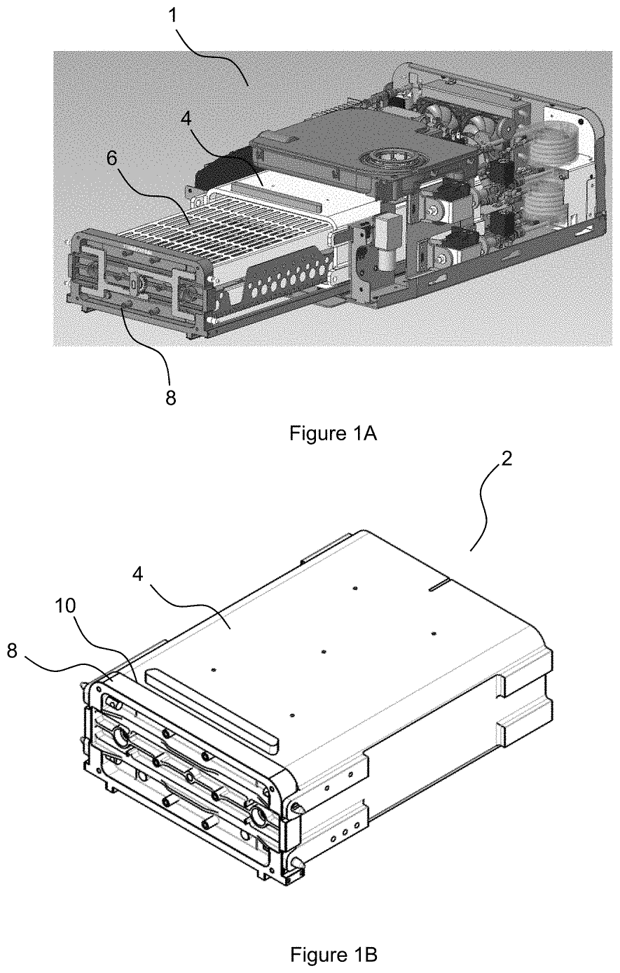

A typical pressure vessel 2 used for autoclaves for the sterilization of instruments is shown in FIGS. 1A and 1B. In this exemplary vessel 2, there is, among other things, a chamber 4 within which the heating and pressurization occurs, and a tray 6 that supports the articles that are to be sterilized. At the outer end of the tray 6 is a door 8. When in use, the tray 6 slides into the chamber 4, and the inner side of the door 8 mates with the front face of the chamber 4 forming a door/chamber interface 10. To withstand the forces created during the operation of the pressure vessel 2, there must be a substantially gas-tight seal at the interface 10. This is typically accomplished by including a seal or gasket 12 at the interface 10.

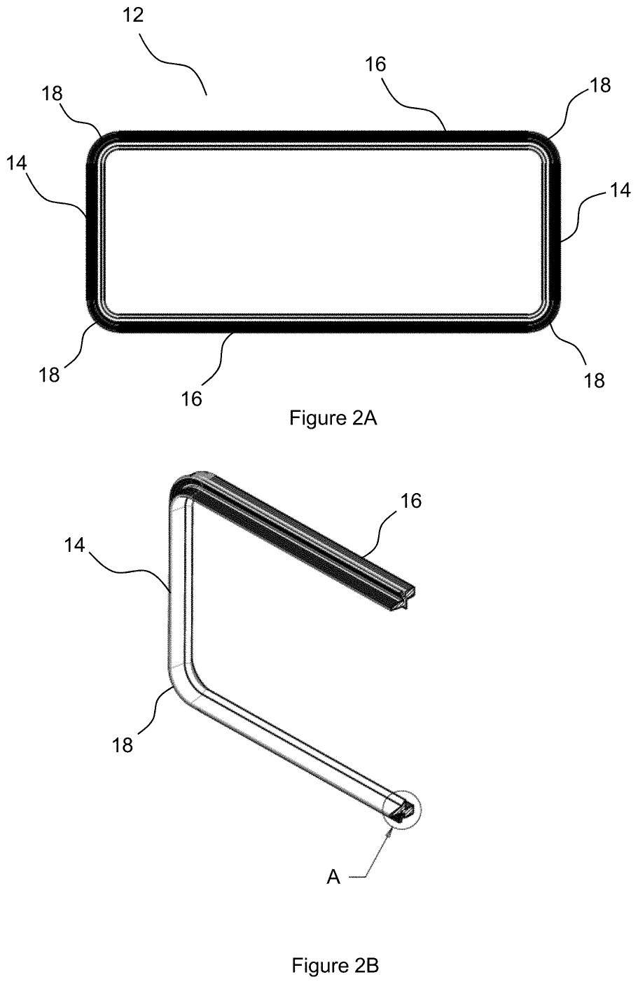

A seal according to one embodiment is shown in FIGS. 2A and 2B. In these Figures, the seal 12 is shown in a substantially rectangular shape that would mate with the door 8 of the exemplary pressure vessel 2 illustrated in FIGS. 1A and 1B. However, it is to be appreciated that the seal 12 can take on any shape necessary to fit the interface between the two structures that are to be sealed. For example, if the door had a square shape, then the seal 12 would be substantially square. If the seal 12 was to be used at the interface between a circular cap and a container, the seal would have a substantially circular shape.

In the embodiment shown in FIGS. 2A and 2B, the seal 12 has two opposing short sides 14 that are substantially vertical when the seal 12 is installed on the door 8 of the pressure vessel 2, and two opposing long sides 16 that are substantially horizontal when the seal 12 is installed on the door 8 of the pressure vessel 2. Connecting the short sides 14 with the long sides 16 are corners 18. The corners 18 may substantially form right angles, however, in the illustrated embodiment, the corners 18 are radiused.

Preferably, the seal 12 is made from an elastomeric material that is soft enough to create a pressure retaining boundary between the door 8 and the chamber 4. The vacuum seal capability of the seal 12 relies on the elasticity of the material to compress against the chamber face 48 and form a gas-tight vacuum seal. The material of the seal 12 should preferably have good resistance to breaking when stretched, some amount of heat resistance, elastic properties, some amount of tear resistance, some amount of abrasion resistance, etc. For example, the material can be natural rubbers, and synthetic rubbers, including saturated and unsaturated rubbers. Some examples include nitrile rubbers, styrene-butadiene rubbers, fluororubbers, silicone, neoprene, and the like.

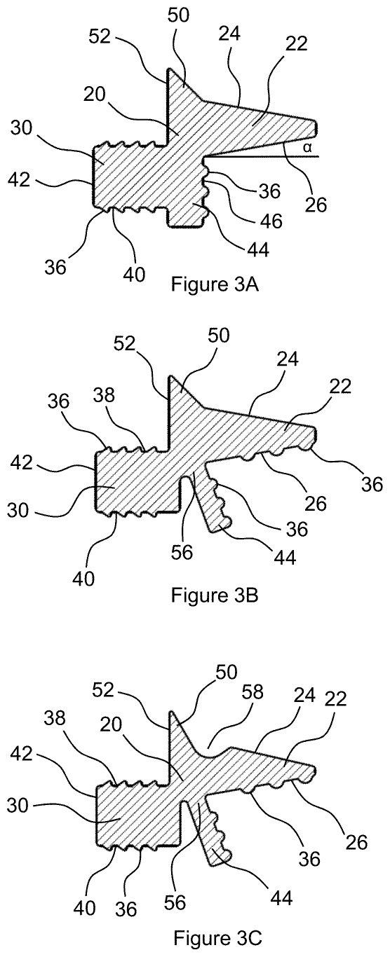

FIGS. 3A-3C illustrate longitudinal cross section views of three different embodiments of the seal 12. For a point of reference, the seals 12 illustrated in these views would be cross sections from the lower long side 16 of the seal shown in FIGS. 2A and 2B.

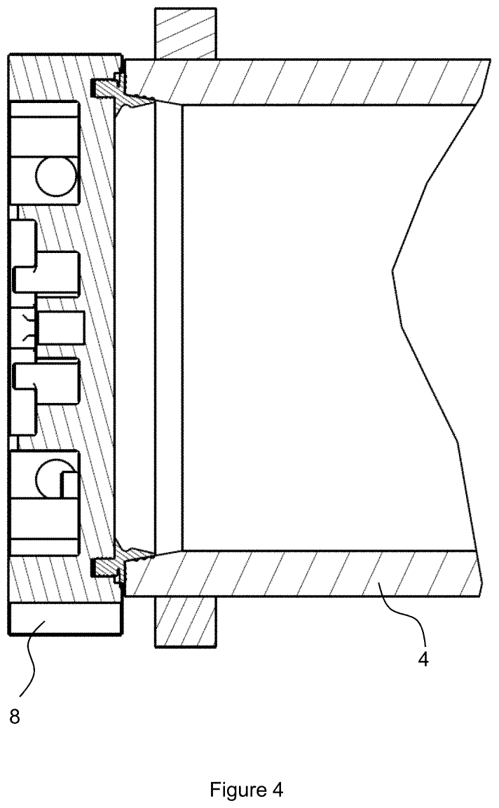

In FIG. 3A, the seal 12 has a main body 20. Extending outward from the main body 20 is a primary sealing lobe 22 having a top surface 24 and a bottom surface 26. When the door of the pressure vessel 2 is closed, this lobe 22 extends into the chamber 4, and abuts the chamber lead-in 28 (See e.g. FIGS. 4-6). Accordingly, the shape of the primary sealing lobe 22, including the angle α of the bottom surface 26 of the primary sealing lobe 22 in relation to the main body 20, may vary based on the use of the seal 12 and the shape of the mating structure. Preferably, the shape, including the angle α, will reflect the structure that the primary sealing lobe 22 will abut. For example, in the exemplary use presented in the present application, the primary sealing lobe has a generally cone-shaped cross section, which creates an angle α at the bottom surface of the primary sealing lobe 22 that matches the chamber mouth lead-in 28. Alternatively, the angle α may be less than the incline angle of the chamber lead-in 28, which would bias the primary sealing lobe 22 toward the chamber lead-in 28. The mating shape of the primary sealing lobe 22 with the chamber mouth lead-in 28 facilitates closing of the door 8, reduces the manual force required to close the door, and generally creates a stronger seal.

Extending outward on the opposing side of the main body 20 from the primary sealing lobe 22 is the door channel lobe 30. This lobe 30, which preferably extends along the length of the seal 12, is generally inserted into a channel 32 around the perimeter of the inner face 34 of the door 8 (See e.g. FIGS. 4-6). Although this lobe 30 is illustrated as having a generally rectangular cross section, the shape of this lobe 30 may vary to reflect the shape of the channel 32 into which the door channel lobe 30 will be inserted. As the door channel lobe 30 is preferably secured in the channel 32 by a pressure fit, the door channel lobe 30 should directly mirror the shape and dimensions of the channel 32.

Optionally, there may be ribs and/or protrusions 36 on the top 38, bottom 40, and/or end 42 of the door channel lobe 30. The ribs 36 may extend along the length of the door channel lobe 24, and may be linear, or extend in other patterns, such as a zig-zag or wave pattern. The protrusions 36 may be randomly or sequentially arranged along the length of the door channel lobe 30. The protrusions 36 can take any shape, e.g. circular, oval, square, etc. The ribs and/or protrusions 36 serve to increase the surface area of the door channel lobe 30, thereby increasing friction that will enhance the pressure fit of the door channel lobe 30 in the channel 32.

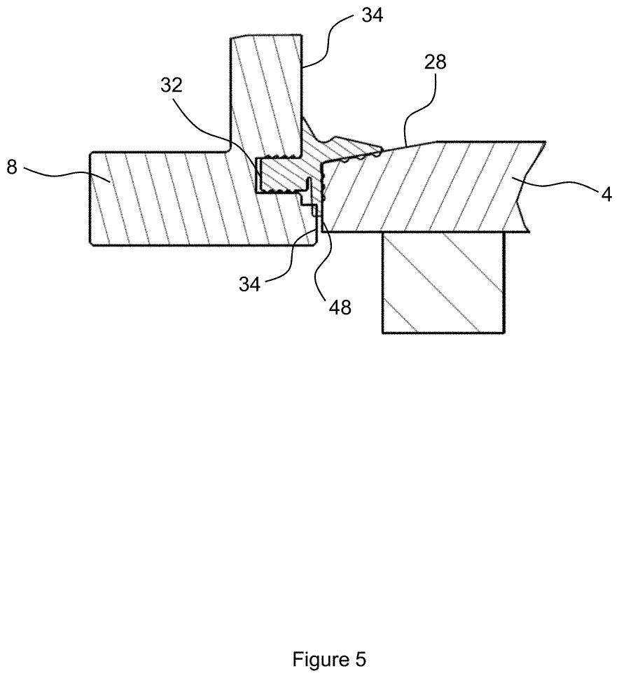

Extending in a downward direction from the main body 20 is a secondary sealing lobe 44. When the door 8 of the pressure vessel 2 is in the closed position (see FIGS. 4 and 5), the outer face 46 of the secondary sealing lobe 44 abuts against the front face 48 of the chamber 4. The length of the secondary sealing lobe 44 can vary, as it may extend from the main body 20 a sufficient distance to engage all or only some of the front face 48 of the chamber 4. As can be seen in FIG. 3A, the outer face 46 of the secondary sealing lobe 44 may have ribs and/or protrusions 36. The nature of the ribs and/or protrusions 36 on the outer face 46 are as discussed above. Ribs and/or protrusions 36 on the outer face of the secondary sealing lobe 44 serve to increase surface area, thereby enhancing the seal between the door 8 and the chamber 4.

Optionally, an additional inner lobe 50 extends upward from the main body 20. Although this lobe 50 is shown as having a triangular shape in the Figures, other shapes may be used. Preferably, the outer face 52 is substantially flat as it abuts the inner face 34 of the door 8 (See FIGS. 4-6). The inner lobe 50 facilitates creating the pressure seal between the door 8 and the chamber 4.

FIG. 3B shows another embodiment of the seal 12 with a set of similar features to the embodiment shown in FIG. 3A. Like features are denoted with the same reference characters. This embodiment reflects the cross section A of the seal 12 shown in FIGS. 2A and 2B.

In this embodiment, the main body 20 of the seal 12 is bifurcated at its lower end, generating a slit between at least part of the secondary sealing lobe 44 and the door channel lobe 20 (See e.g. FIGS. 3B and 3C). In this embodiment, at least a portion of the secondary sealing lobe 44 is separated from the door channel lobe 30. In this embodiment, the separated secondary sealing lobe 44 is connected to the main body 20 via a connection point 56, which effectively serves as a hinge for the secondary sealing lobe 44. Based on the elastic nature of the material that forms the seal 12, and the relative thinness of the connection point 56, the secondary sealing lobe 44 is able to flex forward and backward about the connection point 56 relative to the remainder of the seal 12.

The secondary sealing lobe 44 can be biased outward toward the primary sealing lobe 22 as shown in FIGS. 3B and 3C. This added mobility allows the secondary sealing lobe 44 to be drawn into the front face 48 of the chamber 4 during the vacuum phase of the pressure vessel 2. This flexure of the secondary sealing lobe 44 also reduces the force required to compress the seal 12 and reduces the force necessary to close the door 8.

In addition, in the embodiment shown in FIG. 3B, the bottom surface 26 of the primary sealing lobe 22 is shown with ribs and/or protrusions 36. Although not shown in FIG. 3A, it is to be understood that the embodiment of the seal 12 illustrated in FIG. 3A may also include ribs and/or protrusions 36 on the bottom surface 26 of the primary sealing lobe 22. The ribs and/or protrusions 36 are as discussed above, and serve to create additional friction to enhance the engagement of the bottom surface 26 with the chamber lead-in 28, and generally facilitates creating the seal between the door 8 and the chamber 4.

FIG. 3C shows another embodiment of the seal 12 with a set of similar features to the embodiment shown in FIG. 3B. Again, like features are denoted with the same reference characters.

In this embodiment, a portion of the seal material of the main body 20 at a transition point between the primary sealing lobe 22 and the inner lobe 50 has been removed, creating a depression 58, which may extend along some or all of the length of the seal 12. The depression 58 may be absent from the corners 18 of the seal 12. The depression 58 enables some amount of upward flex of the primary sealing lobe 22, which would be beneficial when the door 8 of the pressure vessel 2 is closed and the primary sealing lobe 22 is in contact with the chamber lead-in 28 (see FIGS. 4 and 5). For example, as the door 8 closes and the primarily sealing lobe is inserted into the chamber 4, the bottom surface 26 of the primary sealing lobe 22 runs along the chamber lead in 28. While the angle α is such that the primary sealing lobe 22 generally reflects the incline angle of the chamber lead in 28, the primary sealing lobe 22 may need to flex upward. The depression 58 facilitates the upward flexure of the primary sealing lobe 22. By allowing some flexure of the primary sealing lobe 22, the depression 58 enhances the sealing ability of the primary sealing lobe 22, reduces the door closing force, and may increase the lifespan of the seal 12.

In one embodiment of the seal 12, in which it has a shape as depicted in FIGS. 2A and 2B, when such a seal is used in connection with a pressure vessel 2, the seal 12 may have a different cross section in at least one of the corners 18 than in the long sides 16 and/or short sides 14. Due to the geometry of the seal 12 and chamber 4, the amount of deflection on the seal 12 that occurs during chamber 4 pressurization is generally greater along the long sides 16 and short sides 14 than along the corners 18. Accordingly, the seal 12 on the long sides 16 and/or short sides 14 can have larger dimensions, such as thickness, relative to the corners 18, as this would provide additional material that would strengthen these portions. Alternatively, the long sides 16 and/or the short sides 14 of the seal 12 can be made of a different more rigid material than the corners 18.

FIGS. 4-6 illustrate the embodiment of the seal 12 as shown in FIG. 3C in use with a pressure vessel 2. In FIGS. 4 and 5, a longitudinal cross section of the front portion of the pressure vessel 2 is shown, in which the door 8 is in the closed position. The seal 12 is attached to the door 8 by inserting the door channel lobe 30 into the door channel 32. This places the inner face 52 of the inner lobe substantially flush against the inner face 34 of the door 8.

As the door is being closed, the bottom surface 26 of the primary sealing lobe 22 contacts and rides along the chamber lead-in surface 28, which may cause the primary sealing lobe 22 to flex upward to ensure snug contact. However, the elasticity of the material of the seal 12 will force the primary sealing lobe 22 downward toward the chamber lead-in surface 28. In addition, the outer face 46 of the secondary sealing lobe 44 contacts the front face 48 of the chamber 4, which forces the secondary sealing lobe 44 to flex backward toward the inner face 34 of the door 8. When in the fully closed position (See FIGS. 4 and 5), the secondary sealing lobe 44 is compressed and pinned between the inner face 34 of the door 8 and the front face 48 of the chamber 4. Collectively, the seal 12 has filled the gap at the interface 10 between the chamber 4 and the door 8 of the pressure vessel 2.

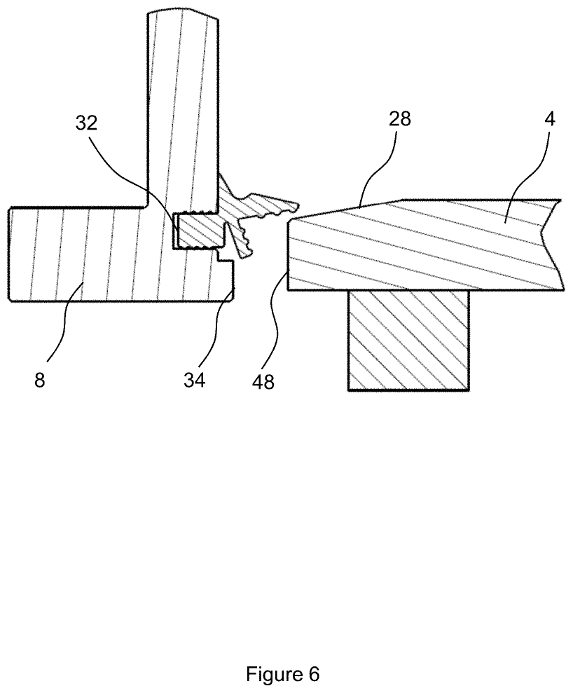

FIG. 6 illustrates that when the door 8 is opened, such as after the sterilization cycle of the pressure vessel 2, the seal 12 returns to its native form.

What is claimed is:

1. A seal for sealing the interface between abutting first and second members, the seal comprising:
a main body;
a channel lobe extending outward from the main body, the channel lobe shaped for nesting in a channel of the first member;
a primary sealing lobe extending outward from the side of the main body facing the second member, the primary sealing lobe extending into and contacting an inner surface of the second member; and
a secondary sealing lobe extending downward from the main body toward an exterior of the interface, a first face of a distal end of the secondary sealing lobe contacting a front face of the first member, and a second face of the distal end of the secondary sealing member contacting a front face of the second member.

2. The seal of claim 1, wherein the main body is at least partially bifurcated, creating a slit between a portion of the channel lobe and a portion of the secondary sealing lobe.

3. The seal of claim 2, wherein when the first member and the second member are in a non-abutting state, the secondary sealing lobe is biased toward the primary sealing lobe.

4. The seal of claim 1, further comprising:
an inner lobe extending outward from the main body toward the interior of the interface, a first face of the inner lobe contacting the front face of the first member.

5. The seal of claim 4, wherein there is a depression in the main body at a transition point between the primary sealing lobe and the inner lobe.

6. The seal of claim 5, wherein when the first member and second member are in an abutting state, the depression permits flexure of the primary sealing lobe toward the inner lobe.

7. The seal of claim 1, wherein there are ribs and/or protrusions on a surface of one or more of the channel lobe, the primary sealing lobe, the secondary lobe, or the inner lobe.

8. The seal of claim 1, wherein the seal is made of an elastomeric material.

9. The seal of claim 1, wherein the seal is contiguous to seal a perimeter of the interface between the first and second members.

10. The seal of claim 1, wherein the seal has an annular shape, a substantially rectangular shape, or a substantially square shape.

11. The seal of claim 1, wherein the primary sealing lobe has a cone-shaped cross section.

12. A seal for sealing the interface between abutting first and second members, the seal comprising:
a main body;
a channel lobe extending outward from the main body, the channel lobe shaped for nesting in a channel of the first member;
a primary sealing lobe extending outward from the side of the main body facing the second member, the primary sealing lobe extending into and contacting an inner surface of the second member;
a secondary sealing lobe extending downward from the main body toward an exterior of the interface, a first face of a distal end of the secondary sealing lobe contacting a front face of the first member, and a second face of the distal end of the secondary sealing member contacting a front face of the second member; and
an inner lobe extending outward from the main body toward the interior of the interface, a first face of the inner lobe contacting the front face of the first member,
wherein the main body is at least partially bifurcated, creating a slit between a portion of the channel lobe and a portion of the secondary sealing lobe; and
there is a depression in the main body at a transition point between the primary sealing lobe and the inner lobe.

13. An autoclave comprising the seal of claim 1 at an interface between a door and a pressurization chamber.
